(19) United States
(12) Patent Application Publication
(10) Pub. No.: US 2022/0225639 A1
(43) Pub. Date: Jul. 21, 2022

(54) IMMUNE-STIMULATING COMPOSITIONS FOR AQUACULTURE SPECIES

(71) Applicant: MIREXUS BIOTECHNOLOGIES INC., Guelph (CA)

(72) Inventors: Jondavid DE JONG, Guelph (CA); Stephanie DEWITTE-ORR, Waterloo (CA); Kristof JENIK, Waterloo (CA); Tamiru ALKIE, Winnipeg (CA)

(21) Appl. No.: 17/610,356

(22) PCT Filed: Feb. 26, 2020
(86) PCT No.: PCT/CA2020/050254

§ 371 Date: Nov. 10, 2021

Related U.S. Application Data
(60) Provisional application No. 62/846,491, filed on May 10, 2019.

(51) Int. Cl. A23K 20/153; A23K 20/163; A23K 40/30; A23K 50/80; A61K 31/7105; A61K 31/711; A61K 39/39; A61K 9/14

(52) U.S. Cl. CPC A23K 20/153; A23K 20/163; A23K 40/30; A61K 9/14; A61K 31/7105; A61K 31/711; A61K 39/39; A23K 50/80

(57) ABSTRACT

The present invention relates to immune-stimulating compositions comprising an immunestimulating compound covalently or non-convalently linked to glycogen-based nanoparticles. The immune stimulating compound may be a TLR agonist, dietary nucleotide, or combination thereof. The compositions can elicit a stronger, more robust, localized (intestine) and systemic immune response when delivered orally to aquatic species, than non-glycogen bound agonists and/or molecules.

This application claims priority from U.S. Application No. 62/846,891 filed May 10, 2019 which is incorporated herein by reference.

### FIELD OF THE INVENTION

The present invention relates to immune stimulation for aquaculture species.

### BACKGROUND OF THE INVENTION

The aquaculture industry has a global value of $170 billion and is expected to grow by over 50% by 2030. Protection of aquaculture species from bacterial and viral infection is paramount for a sustainable, resilient aquaculture industry. While there has been some success with bacterial and viral vaccination of aquaculture species, protection levels are far below those seen with human and mammalian vaccine strategies, and the industry needs novel solutions to combat infection. Most viral vaccine efforts involve injection of fish with live attenuated or heat/chemically killed virus, along with an adjuvant. Vaccines are generally administered to fish by injection, which is stressful for the fish, can injure the fish if done improperly and may not be possible for small juvenile fish. Further, administering by injection is labour intensive and thus expensive.

### SUMMARY OF INVENTION

In one aspect, there is provided an immunostimulant for oral administration to aquaculture species comprising a polysaccharide nanoparticle covalently or non-covalently linked to an immune-stimulating compound. In one aspect, there is provided an orally administrable immunostimulant for aquaculture species comprising an immune-stimulating compound covalently or non-covalently linked to glycogen-based polysaccharide nanoparticles having a molecular weight of 10⁶ to 10⁷ Da comprising α-D glucose chains, having an average chain length of 11-12, with 1-4 linkage and branching point occurring at 1→6 and with a branching degree of between 6% and 13%.

The immune-stimulating compound may be selected from: double-stranded RNA, double-stranded DNA and single-stranded RNA, single-stranded DNA, and synthetic analogs thereof. In one embodiment, the immune-stimulating compound is poly IC.

The immune-stimulating compound may be one or more nucleotides.

The immune-stimulating compound can comprise between about 50% and 600% by weight relative to the polysaccharide nanoparticles.

In one embodiment, the immunostimulant comprises particles having an average particle diameter of between about 10 nm and 500 nm.

In one embodiment, the immune-stimulating compound is covalently linked to the nanoparticles. In another embodiment, the nanoparticles are cationic and the immunestimulating compound is non-covalently linked to the nanoparticles through electrostatic interactions. The cationic nanoparticles may be amine-modified, in some embodiments with a short-chain quaternary ammonium compound comprising at least one alkyl moiety having from 1 to 16 carbon atoms, unsubstituted or substituted with one or more N, O, S, or halogen atoms.

The nanoparticles can further be covalently linked to one or more small molecules for directing the nanoparticles to a type of cell or cellular compartment.

In one embodiment, the immunostimulant is in the form of a powder.

In another aspect, there is provided a vaccine adjuvant for aquaculture species comprising the orally administrable immunostimulant as described.

In another aspect, there is provided a food source or food ingredient for aquaculture species comprising the orally administrable immunostimulant as described.

In another aspect, there is provided a coating for a food source or food ingredient for aquaculture species comprising the orally administrable immunostimulant as described.

Also provided is a method of stimulating an innate immune response in an aquaculture species comprising administering to the aquaculture species orally or via immersion bath a therapeutically effective amount of an immunostimulant comprising: an immunestimulating compound covalently or non-covalently linked to glycogen-based polysaccharide nanoparticles having a molecular weight of 10⁶ to 10⁷ Da comprising α-D glucose chains, having an average chain length of 11-12, with 1→4 linkage and branching point occurring at 1→6 and with a branching degree of between 6% and 13%.

The method may be for preventing or treating a viral, bacterial, parasitic or fungal infection in the aquaculture species. In one embodiment, the aquaculture species is a teleost fish.

In one embodiment, the immunostimulant is administered in conjunction with a vaccine.

In one embodiment, the method upregulates expression of one or more of IFN1, VIG-3 and MX1 in the aquaculture species.

### BRIEF DESCRIPTION OF FIGURES

FIG. 1 shows levels of interferon 1 (IFN1) expression at the transcript level following treatment with poly IC or poly IC linked to phytoglycogen nanoparticles (poly IC:PhG). RTgutGC cells were treated with media alone (CTRL), 1 μg/mL of poly IC and 1 μg/mL of poly IC covalently linked to 6.875 μg/mL PhG (poly IC:PhG) for 24 hours. IFN1 transcript levels were measured using qRT-PCR.

FIG. 2 shows levels of vig3 expression at the transcript level following treatment with poly IC or poly IC:PhG. RTgutGC cells were treated with media alone (CTRL), 1 μg/mL of poly IC and 1 μg/mL of poly IC covalently linked to 6.875 μg/mL PhG (poly IC:PhG) for 24 hours. Vig3 transcript levels were measured using qRT-PCR.

FIG. 3 shows the ability of cationized phytoglycogen nanoparticles (Cat-PhG) to bind poly IC as demonstrated by electrophoretic mobility shift assay (EMSA). Three (3) micrograms of poly:IC was mixed with 2-fold dilution series of Cat-PhG harbouring a 0.88 degree of substitution (ds). After a 20-minute incubation at room temperature, samples were separated on a 1% agarose gel, stained with ethidium bromide, and subsequently imaged under UV light. Shown are the EMSA gels for Cat-PhG-0.88 demonstrating maximum loading ˜6:1 (poly:IC:PhG w/w).

FIG. 4 shows PhG enhances uptake and intracellular stability of TLR 3 agonist in rainbow trout gut cells (RT-gut). Per Example 7, low molecular weight poly:IC was labelled with a fluorescent dye, then left as “free” poly:IC or coupled with Cat-PhG (0.38) at a 1:1 (w/w) ratio. Free poly:IC or PhG coupled poly:IC was added to RT-gut cells at a concentration of 1 ug/ml and incubated at 4° C. or 20° C. for 4 hours. Cells were imaged via fluorescent microscopy, and average intracellular fluorescence intensity was measured from 5 randomly selected cells.

FIG. 5 shows transcription levels of IFN1, MX1, and Vig-3, as measured by qRT-PCR, in the proximal intestine at 24 hours following oral gavage of fish feed mixed with 250 μg of high molecular weight (HMVV) poly I:C ionically bound to Cat-PhG at a 1:1 (w/w) ratio (Nano-PIC), or 250 μg of HMW poly I:C (PIC). Nanoparticle bound HMW poly I:C caused greater localized immune stimulation when compared to the “free” HMW poly I:C at 24 hours post-feeding.

FIG. 6 shows transcription levels of IFN1, MX1, and Vig-3, as measured by qRT-PCR, in the median intestine at 24 hours following oral gavage of fish feed mixed with 250 μg of HMW poly I:C ionically bound to Cat-PhG at a 1:1 (w/w) ratio (Nano-PIC), or 250 μg of HMW poly I:C (PIC). Nanoparticle bound HMW poly I:C caused greater localized immune stimulation when compared to the “free” HMW poly I:C at 24 hours post-feeding.

FIG. 7 shows transcription levels of IFN1, MX1, and Vig-3, as measured by qRT-PCR, in the distal intestine at 24 hours following oral gavage of fish feed mixed with 250 μg of HMW poly I:C ionically bound to Cat-PhG at a 1:1 (w/w) ratio (Nano-PIC), or 250 μg of HMW poly I:C (PIC). Nanoparticle bound HMW poly I:C caused greater localized immune stimulation when compared to the “free” HMW poly I:C at 24 hours post-feeding.

FIG. 8 shows transcription levels of IFN1, MX1, and Vig-3, as measured by qRT-PCR, in the proximal intestine at 48 hours following oral gavage of fish feed mixed with 250 μg of HMW poly I:C ionically bound to Cat-PhG at a 1:1 (w/w) ratio (Nano-PIC), or 250 μg of HMW poly I:C (PIC). Nanoparticle bound HMW poly I:C caused greater localized immune stimulation when compared to the “free” HMW poly I:C at 48 hours post-feeding.

FIG. 9 shows transcription levels of IFN1, MX1, and Vig-3, as measured by qRT-PCR, in the median intestine at 48 hours following oral gavage of fish feed mixed with 250 μg of HMW poly I:C ionically bound to Cat-PhG at a 1:1 (w/w) ratio (Nano-PIC), or 250 μg of HMW poly I:C (PIC). Nanoparticle bound HMW poly I:C causes greater localized immune stimulation when compared to the “free” HMW poly I:C at 48 hours post-feeding.

FIG. 10 shows transcription levels of IFN1, MX1, and Vig-3, as measured by qRTPCR, in the distal intestine at 48 hours following oral gavage of fish feed mixed with 250 μg of HMW poly I:C ionically bound to Cat-PhG at a 1:1 (w/w) ratio (Nano-PIC), or 250 μg of HMW poly I:C (PIC). Nanoparticle bound HMW poly I:C caused greater localized immune stimulation when compared to the “free” HMW poly I:C at 48 hours post-feeding.

FIG. 11 shows transcription levels of IFN1, MX1, and Vig-3, as measured by qRTPCR, in the head kidney at 24 hours following oral gavage of fish feed mixed with 250 μg of HMW poly I:C ionically bound to Cat-PhG at a 1:1 (w/w) ratio (Nano-PIC), or 250 μg of HMW poly I:C (PIC). Nanoparticle bound HMW poly I:C caused greater systemic immune stimulation when compared to the “free” HMW poly I:C at 24 hours post-feeding.

FIG. 12 shows transcription levels of IFN1, MX1, and Vig-3, as measured by qRTPCR, in the head kidney at 48 hours following oral gavage of fish feed mixed with 250 μg of HMW poly I:C ionically bound to Cat-PhG at a 1:1 (w/w) ratio (Nano-PIC), or 250 μg of HMW poly I:C (PIC). Nanoparticle bound HMW poly I:C caused greater systemic immune stimulation when compared to the “free” HMW poly I:C at 48 hours post-feeding.

FIG. 13 shows transcription levels of MX1 and Vig-3, as measured by qRT-PCR, in the proximal intestine at 24 hours following feeding with pellets formulated with 250 μg of high molecular weight (HMVV) poly I:C ionically bound to Cat-PhG at a 1:1 (w/w) ratio (PIC+NDX), or 250 μg of HMW poly I:C (PIC). Nanoparticle bound HMW poly I:C caused greater localized immune stimulation when compared to the “free” HMW poly I:C at 24 hours post-feeding.

FIG. 14 shows transcription levels of MX1 and Vig-3, as measured by qRT-PCR, in the medial intestine at 48 hours following feeding with pellets formulated with 250 μg of high molecular weight (HMVV) poly I:C ionically bound to Cat-PhG at a 1:1 (w/w) ratio (PIC+NDX), or 250 μg of HMW poly I:C (PIC). Nanoparticle bound HMW poly I:C caused greater localized immune stimulation when compared to the “free” HMW poly I:C at 48 hours post-feeding.

FIG. 15 shows transcription levels of MX1 and Vig-3, as measured by qRT-PCR, in the head kidney at 48 hours following feeding with pellets formulated with 250 μg of high molecular weight (HMW) poly I:C ionically bound to Cat-PhG at a 1:1 (w/w) ratio (PIC+NDX), or 250 μg of HMW poly I:C (PIC). Nanoparticle bound HMW poly I:C caused greater systemic immune stimulation when compared to the “free” HMW poly I:C at 24 hours post-feeding.

FIG. 16 shows transcription levels of MX1 and Vig-3, as measured by qRT-PCR, in the gills at 24 hours following feeding with pellets formulated with 250 μg of high molecular weight (HMW) poly I:C ionically bound to Cat-PhG at a 1:1 (w/w) ratio (PIC+NDX), or 250 μg of HMW poly I:C (PIC). Nanoparticle bound HMW poly I:C caused greater systemic immune stimulation when compared to the “free” HMW poly I:C at 24 hours post-feeding.

FIG. 17 shows RNAseiii digestion of “free” poly I:C or poly I:C bound to Cat-PhG at various ratios. Five (5) μg of HMW poly I:C was left unbound, or allowed to bind to Cat-PhG at ratios of 2:1, 1:1, and 0.5:1 phytoglycogen:poly I:C (w/w), then subjected to RNAse iii digestion and samples were separated via agarose gel and visualized with nucleic acid stain.

FIG. 18 shows transcription levels of IFN1 and MX1 as measured by qRTPCR, in the Rainbow Trout intestinal epithelial cells (RT-GUT) mock-treated (NDX) or treated with “free” IMP (IMP), “free” poly I:C (Cont), or IMP covalently bound to aminated phytoglycogen nanoparticles (IMP+NDX). Nanoparticle bound IMP generated innate immune stimulation (MX1) at concentrations where free IMP has no effect.

### DETAILED DESCRIPTION

As used herein “immunotherapy” refers to treating or preventing disease by inducing, enhancing or suppressing an immune response. As used herein “stimulating an immune response” refers to inducing, enhancing or amplifying an immune response and a compound or composition for stimulating an immune response is referred to as an immunostimulant.

In one aspect, there is provided compounds and compositions for use in stimulating an innate immune response. As used herein, “stimulating an innate immune response” or grammatical variations thereof refers to upregulating one or more genes associated with an innate immune response, for example (and without being limited thereto) VIG3, IFN, CXCL10, MX1 and IFIT1.

As used herein “aquaculture species” refers to a farmed aquatic organism, in one embodiment a non-plant or non-algae organism, which may be a fish, mollusc or crustacean, including e.g. teleost fish, non-teleost fish, shrimp, clams, mollusks, oysters and mussels.

A challenge with oral administration of vaccines or immunostimulants is that immunestimulating compounds may be inactivated by the acidic environment or enzymatic activity in the gut thereby precluding absorption from the intestine of active compound.

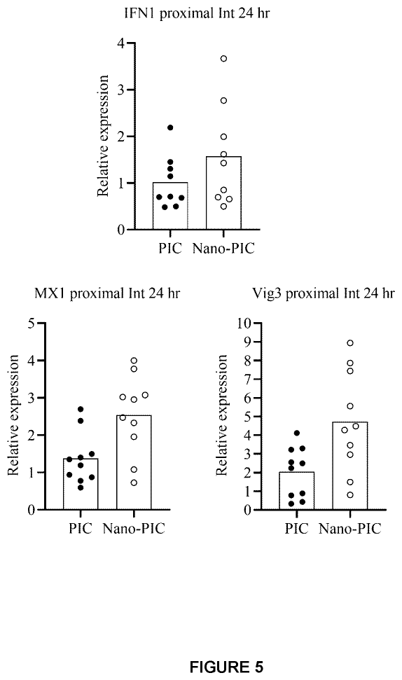

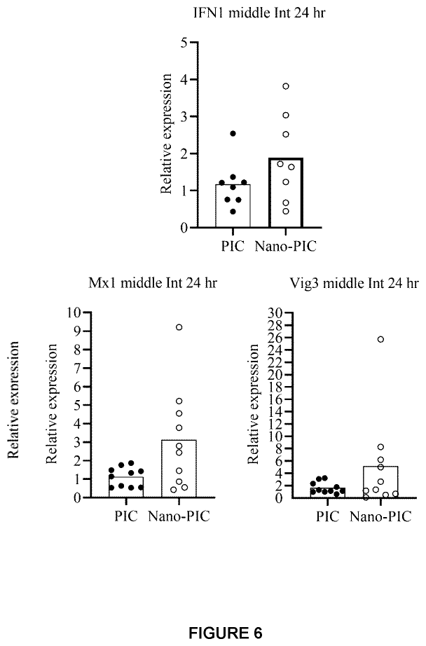

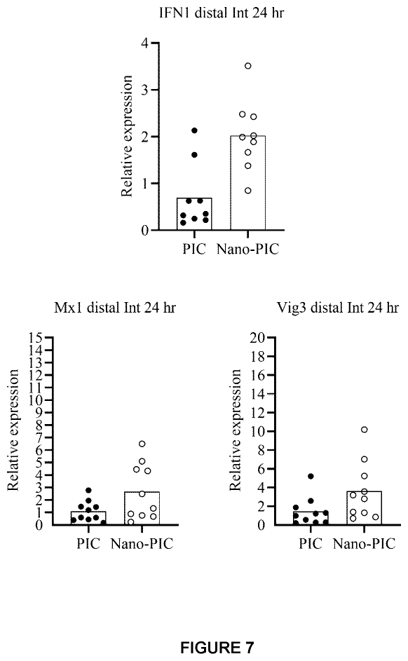

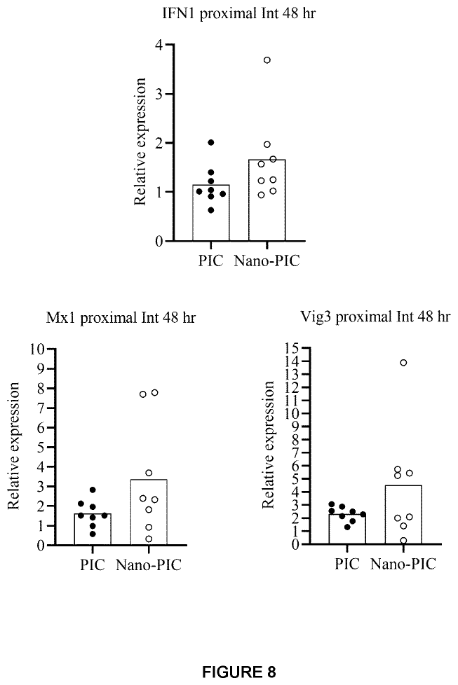

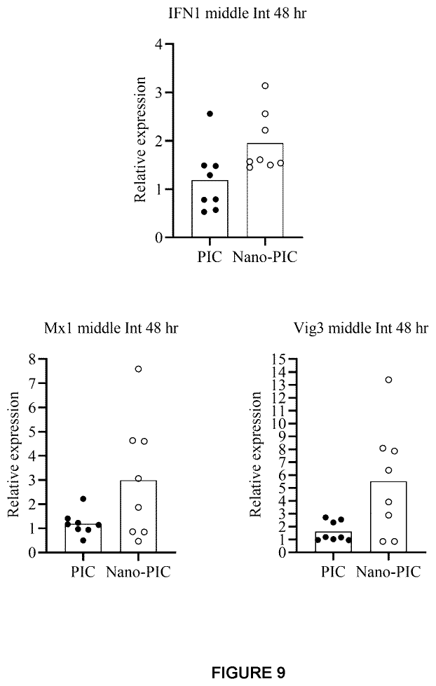

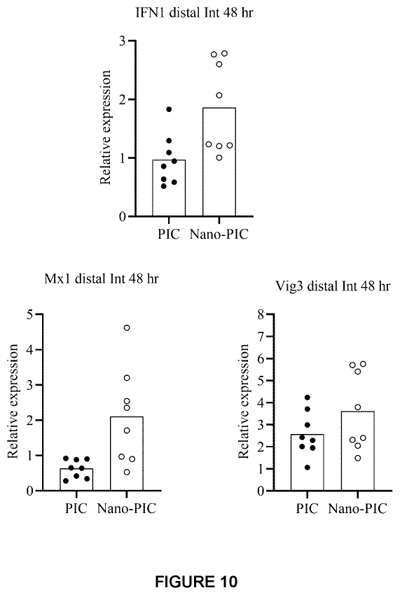

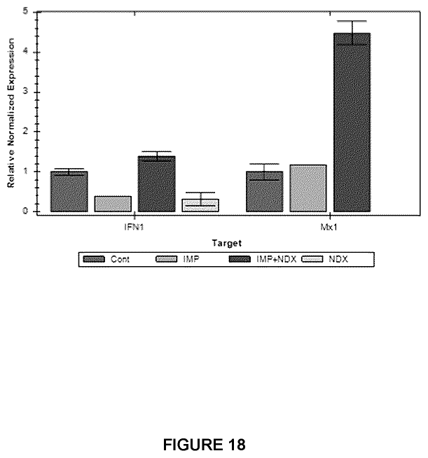

The present inventors have demonstrated that being bound to glycogen nanoparticles (whether covalently or non-covalently) as exemplified herein increases the potency of TLR based (for example poly I:C) or non-TLR based (for example IMP) innate immune stimulants, when orally delivered to aquaculture species (FIGS. 5-16, 18).

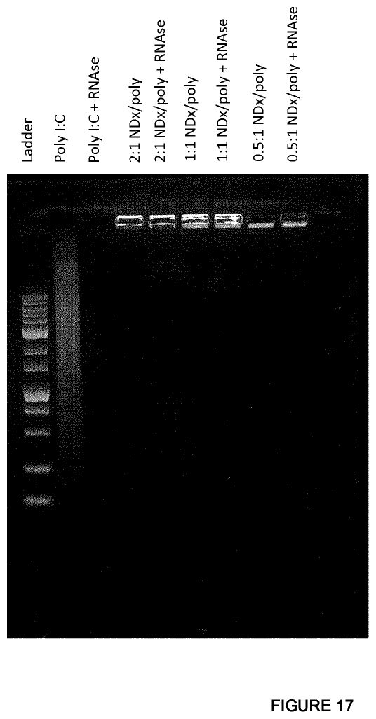

As demonstrated in the Examples, the compounds and compositions remain immune stimulatory after extrusion and baking when formulated in fish feed, and additionally protect nucleic acid-based ligands from enzymatic digestion (FIG. 17).

As demonstrated in the Examples, the present inventors have found that the immunostimulants described herein, surprisingly, induce a robust immune response across the full-length of the intestine after oral delivery.

Even more surprisingly, the present inventors have found, as exemplified herein, that the described immunostimulants induce a robust systemic immune stimulation, after oral delivery.

In one aspect, there is provided novel methods and vehicles for delivery of immunestimulating compounds.

In various embodiments, the immune stimulating compound is not directed to a specific pathogen but stimulates the innate immune response. The immunostimulants as described herein are useful for increasing the resilience of aquaculture species to stress, in the face of exposure or risk of exposure to a pathogen and/or as a vaccine adjuvant.

The efficacy of vaccine can be increased by the co-delivery of a TLR agonist (see for example, Nishizawa et al, 2009, which teaches that fish can be protected from Viral Nervous Necrosis virus (VNNV) infection when Poly I:C is delivered via injection, followed by injection of a VNNV.)

Injection of TLR agonist (Poly I:C, CpG ODN) either in the free form or encapsulated in liposomal nanoparticles has been shown to stimulate the innate immune system (see e.g. EP3164113B1).

While somewhat effective, injection of fish in large scale aquaculture systems is not feasible. Therefore, there exists a need for novel food-based additives that can provide not only localized, but systemic immune stimulation in aquaculture species. The present invention provides a “passive” method of stimulating an innate immune-response in or administering a vaccine to an aquaculture species that does not require injection, in particular, the immunostimulant may be administered orally or via an immersion bath. In a preferred embodiment, the administration is oral administration.

Glycogen and phytoglycogen are composed of molecules of α-D glucose chains having an average chain length of 11-12, with 1→4 linkage and branching point occurring at 1→6 and with a branching degree of about 6% to about 13%. Glycogen and phytoglycogen molecules may be modified as described further below; “glycogen-based polysaccharide” refers to a polysaccharide exhibiting this structure although subject to further modifications.

The yields of most known methods for producing glycogen or phytoglycogen and most commercial sources are highly polydisperse products that include both glycogen or phytoglycogen particles, as well as other products and degradation products of glycogen or phytoglycogen.

As used herein “glycogen nanoparticles” is used to refer to both glycogen and phytoglycogen nanoparticles, however, it will be understood that in a preferred embodiment, phytoglycogen nanoparticles are used. Accordingly, unless specifically and explicitly excluded, it will be understood the embodiments described include nanoparticles manufactured from plant starting materials.

“Glycogen” can include both products derived from natural sources and synthetic products, including synthetic phytoglycogen i.e. glycogen-like products prepared using enzymatic processes on substrates that include plant-derived material e.g. starch.

In one embodiment, monodisperse glycogen nanoparticles are used. In a further preferred embodiment, monodisperse phytoglycogen nanoparticles are used.

Glycogen nanoparticles as described herein are non-toxic, have no known allergenicity, and can be degraded by glycogenolytic enzymes. The products of enzymatic degradation are non-toxic molecules of glucose. In one embodiment, glycogen refers to glycogen nanoparticles manufactured according to methods described herein. The described methods enable production of substantially spherical nanoparticles, each of which is a single glycogen molecule.

United States patent application publication no. United States 20100272639 A1, assigned to the owner of the present application and the disclosure of which is incorporated by reference in its entirety, provides a process for the production of glycogen nanoparticles from bacterial and shell fish biomass. The processes disclosed generally include the steps of mechanical cell disintegration, or by chemical treatment; separation of insoluble cell components by centrifugation; elimination of proteins and nucleic acids from cell lysate by enzymatic treatment followed by dialysis which produces an extract containing crude polysaccharides, lipids, and lipopolysaccharides (LPS) or, alternatively, phenol-water extraction; elimination of LPS by weak acid hydrolysis, or by treatment with salts of multivalent cations, which results in the precipitation of insoluble LPS products; and purification of the glycogen enriched fraction by ultrafiltration and/or size exclusion chromatography; and precipitation of glycogen with a suitable organic solvent or a concentrated glycogen solution can be obtained by ultrafiltration or by ultracentrifugation; and freeze drying to produce a powder of glycogen. Glycogen nanoparticles produced from bacterial biomass were characterized by MWt 5.3-12.7×10⁶ Da, had particle size 35-40 nm in diameter and were monodisperse.

Methods of producing monodisperse compositions of phytoglycogen are described in the International patent application entitled “Phytoglycogen Nanoparticles and Methods of Manufacture Thereof”, published under the international application publication no. WO2014/172786, assigned to the owner of the present application, and the disclosure of which is incorporated by reference in its entirety. In one embodiment, the described methods of producing monodisperse phytoglycogen nanoparticles include: a. immersing disintegrated phytoglycogen-containing plant material in water at a temperature between about 0 and about 50° C.; b. subjecting the product of step (a.) to a solid-liquid separation to obtain an aqueous extract; c. passing the aqueous extract of step (b.) through a microfiltration material having a maximum average pore size of between about 0.05 μm and about 0.15 μm; and d. subjecting the filtrate from step c. to ultrafiltration to remove impurities having a molecular weight of less than about 300 kDa, in one embodiment, less than about 500 kDa, to obtain an aqueous composition comprising monodisperse phytoglycogen nanoparticles. The phytoglycogen containing plant material is suitably a cereal and, in one embodiment, corn. In one embodiment, step c. comprises passing the aqueous extract of step (b.) through (c.1) a first microfiltration material having a maximum average pore size between about 10 μm and about 40 μm; (c.2) a second microfiltration material having a maximum average pore size between about 0.5 μm and about 2.0 μm, and (c.3) a third microfiltration material having a maximum average pore size between about 0.05 and 0.15 μm. The method can further include a step (e.) of subjecting the aqueous composition comprising monodisperse phytoglycogen nanoparticles to enzymatic treatment using amylosucrose, glycosyltransferase, branching enzymes or any combination thereof. The method avoids the use of chemical, enzymatic or thermo treatments that degrade the phytoglycogen material. The aqueous composition can further be dried.

In one embodiment, the composition is obtained from sweet corn (Zea mays var. saccharata and Zea mays var. rugosa). In one embodiment, the sweet corn is of standard (su) type or sugary enhanced (se) type. In one embodiment, the composition is obtained from dent stage or milk stage kernels of sweet corn. Unlike glycogen from animal or bacterial sources, use of phytoglycogen eliminates the risk of contamination with prions or endotoxins, which could be associated with these other sources.

The polydispersity index (PDI) of a composition of nanoparticles can be determined by the dynamic light scattering (DLS) technique and, in this embodiment, PDI is determined as the square of the ratio of standard deviation to mean diameter (PDI=(σ/d)2. PDI can also be expressed through the distribution of the molecular weight of polymer and, in this embodiment, is defined as the ratio of Mw to Mn, where Mw is the weight-average molar mass and Mn is the number-average molar mass (hereafter this PDI measurement is referred to as PDI*). In the first case, a monodisperse material would have a PDI of zero (0.0) and in the second case the PDI* would be 1.0.

In some embodiments, monodisperse glycogen nanoparticles having a PDI of less than about 0.3, less than about 0.2, less than about 0.15, less than about 0.10, or less than 0.05 as measured by DLS are used. In some embodiments, monodisperse glycogen nanoparticles having a PDI* of less than about 1.3, less than about 1.2, less than about 1.15, less than about 1.10, or less than 1.05 as measured by SEC MALS are used.

The glycogen nanoparticles may have an average particle diameter of between about 10 nm and about 150 nm, in one embodiment about 30 nm to about 150 nm, in one embodiment about 60 nm to about 110 nm, and in other embodiments, about 40 nm to about 140 nm, about 50 nm to about 130 nm, about 60 nm to about 120 nm, about 70 nm to about 110 nm, about 80 nm to about 100 nm, about 10 nm to about 30 nm. Once bound to the immune-stimulating compound, the resulting particles may have an average particle diameter of between about 10 nm and about 500 nm, in one embodiment, between about 200 nm and about 400 nm.

In one embodiment, there is provided an immunostimulant for aquaculture species that comprises, consists essentially of, or consists of a composition of glycogen nanoparticles covalently or non-covalently linked to an immune-stimulating compound.

As used herein, “covalently linked” refers to a link via covalent bond, whether directly or via a linker.

As used herein, “non-covalently linked” refers to all non-covalent interactions including electrostatic interactions, hydrophobic interactions, Van der Waals forces and combinations thereof.

Chemical Modification of Glycogen Nanoparticles

To impart specific properties to glycogen nanoparticles, they can be chemically modified via numerous methods common for carbohydrate chemistry.

The resulting products are referred to herein as functionalized or modified nanoparticles or derivatives. Functionalization can be carried out on the surface of the nanoparticle, or on both the surface and the interior of the particle, but the structure of the glycogen molecule as a single branched homopolymer is maintained. In one embodiment, the functionalization is carried out on the surface of the nanoparticle. As will be understood by those of skill in the art, chemical modifications should be non-toxic and generally safe for consumption by aquaculture species.

In some embodiments of the present invention, it is advantageous to change the chemical character of glycogen from its hydrophilic, slightly negatively charged native state to be positively charged, or to be partially or highly hydrophobic. J. F Robyt, Essentials of Carbohydrate Chemistry, Springer, 1998; and M. Smith, and J. March, March's Advanced Organic Chemistry: Reactions, Mechanisms, and Structure Advanced Organic Chemistry, Wiley, 2007 provides certain examples of chemical processing of polysaccharides.

Various derivatives can be produced by chemical modification of hydroxyl groups on glycogen, through one or more functionalization steps. Such functional groups include, but are not limited to, nucleophilic and electrophilic groups, acidic and basic groups, e.g., carboxyl groups, amine groups, thiol groups, and aliphatic hydrocarbon groups such as alkyl, vinyl and allyl groups.

In one embodiment, the functionalized nanoparticles are modified with amino groups, which can be primary, secondary, tertiary, or quaternary amino groups, including quaternary ammonium compounds of varying chain lengths. The short-chain quaternary ammonium compound includes at least one alkyl moiety having from 1 to 27 carbon atoms, unsubstituted or substituted with one or more non-carbon heteroatoms (e.g. N, O, S, or halogen).

In certain embodiments, the nanoparticles described are functionalized via glycidyltrimethylammonium chloride (GTAC) to render an overall positive charge. In certain embodiments, two or more different chemical compounds are used to produce multifunctional derivatives.

To perform direct conjugation reactions between native glycogen nanoparticles under aqueous conditions, water-soluble chemicals with reactive or activated functionalities (e.g. epoxide or anhydride, pH 8-11) are often necessary. To maintain stability of the glycogen nanoparticles, solution pH is preferably slightly basic, optimally between 8 and 9. As the hydroxyl moieties of the native glucose subunits are not sufficiently reactive (i.e. deprotonated) under these conditions, a significant excess of reagent may be required to obtain appreciable functionalization. Although some reactions (e.g. with alkyl halides) are best conducted in organic solvents such as dimethyl sulfoxide (DMSO) or dimethyl formamide (DMF) to improve reagent solubility and homogeneity, derivatization in aqueous environment is preferable, as these reaction conditions are generally mild and impart low to minimal toxicity.

An alternative chemical modification strategy involves activation of glycogen by appending a functionalized linker or conducting a functional group interconversion of the hydroxyl group, to a more chemically active group. This can be performed in aqueous or organic media, offering the advantage of higher chemical selectivity and efficiency. It is possible to isolate the activated glycogen precursors (e.g. aminated, carboxylated) which can then be coupled with a suitable reagent.

By way of example, the simplest approach is the introduction of carbonyl groups by selective oxidation of glucose hydroxyl groups at positions of C-2, C-3, C-4 and/or C-6. There is a wide spectrum of redox agents which can be employed, such as persulfate, periodate, bromine, acetic anhydride, Dess-Martin periodinane, TEMPO (2,2,6,6-Tetramethylpiperidin-1yl)oxyl), etc.

Glycogen nanoparticles functionalized with carboxylate groups are readily reactive towards compounds bearing primary or secondary amine groups. The coupling of these two partners (e.g. through EDC coupling chemistry) results in the formation of amides. This chemistry could also be employed in the reverse direction: reacting amine functionalized glycogen with carboxylate-containing compounds.

For the preparation of primary, secondary or tertiary amino-functionalized nanoparticles, one method of the current invention utilizes the reaction of native glycogen with 2-aminoalkyl halides or hydrogen sulfate. Treatment of the glycogen under basic (pH 9-12) conditions with aminoalkyl substrates results in a nucleophilic substitution reaction, displacing the halide or hydrogen sulfate leaving group. As a result, the glycogen is aminoalkylated (e.g. primary, secondary, or tertiary aminated with an O-alkyl linker). The reaction can be performed at a variety of temperatures (25-90° C.) and aminoalkylating agents of varying chain lengths or leaving groups.

For the quaternary ammonium modification of glycogen, the native nanoparticles are reacted with a variety of 3-chloro-2-hydroxypropyltrialkylammonium chloride reagents, which exist in an epoxide-chlorohydrin equilibrium, depending on solution pH. Under the basic conditions of the reaction performed herein (pH 9-12), the quaternary ammonium reagents are in the epoxide form, which react readily by base-catalyzed ring-opening with the glycogen. The resulting products are 3-(trimethylammonio)-2-hydroxyprop-1-yl or 3-(N-alkyl-N, Ndimethylammonio)-2-hydroxyprop-1-yl glycogen, where in the latter case, the alkyl groups are long-chain alkyl groups including lauryl (C12), cocoalkyl, (C8-C18), and stearyl (C12-C27).

The above quaternary ammonium modified glycogen can then be further reacted with various alkyl, benzyl, or silyl halides to afford nanoparticles bearing both hydrophilic (cationic, quaternary ammonium) and hydrophobic functionalities.

Another route to primary amination of glycogen includes a two-step sequence involving imides. Native glycogen is first reacted under basic conditions with an imide-containing epoxide or alkyl halide, by the chemistry described above, to provide the corresponding (N-imidyl) protected aminoalkyl glycogen (Eq. 1). Depending on the nature of the imide reagent used, the length of the O-alkyl tether and substituents (e.g. imide bearing various alkyl/aryl cyclic or acyclic groups) may be tailored. The N-imidyl group on this product can then be removed by one of several conditions (reducing agent followed by acetic acid at pH 5, aqueous hydrazine hydrate, methylamine, etc.) to afford primary aminoalkylated glycogen nanoparticles (Eq. 2).

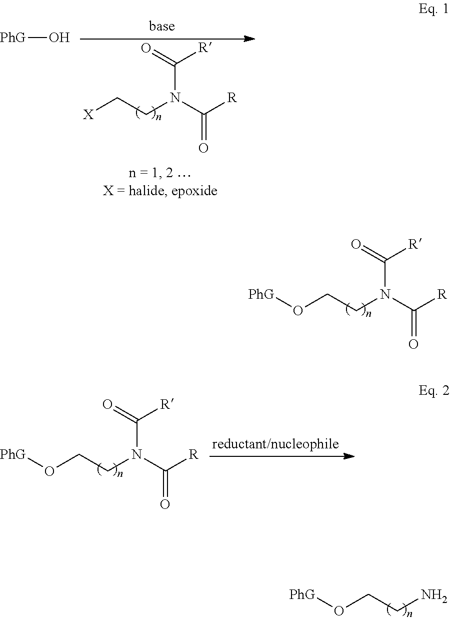

Reductive amination of the nanoparticles can be also achieved by the following two step process. First step is cyanoalkylation, i.e., converting hydroxyls into O-cyanoalkyl groups by reaction with bromoacetonitrile or acrylonitrile. In the second step, the cyano groups are reduced with metal hydrides (borane-THF complex, LiAlH₄, etc).

Amino-functionalized nanoparticles are amenable to further modifications. Amino groups are reactive to carbonyl compounds (aldehydes and ketones), carboxylic acids and their derivatives, (e.g. acyl chlorides, esters), succinimidyl esters, isothiocyanates, sulfonyl chlorides, etc.

Various modifications are exemplified in Example 2.

Covalent and Non-Covalent Linking of Active Agents to Glycogen Nanoparticles

In one embodiment, there is provided an immunostimulant comprising glycogen nanoparticles linked to at least one molecule that induces, enhances or amplifies an immune response in a subject. In one embodiment, the molecule that induces, enhances or amplifies the immune response is covalently linked to the glycogen nanoparticles. In another embodiment, the glycogen nanoparticles are cationized and the molecule that induces, enhances or amplifies the immune response is linked to the cationized glycogen nanoparticles via non-covalent interactions, in one embodiment via ionic bonding.

A chemical compound bearing a functional group capable of binding to carbonyl-, cyanate-, imidocarbonate or amino-groups can be directly attached to functionalized glycogen nanoparticles. However, for some applications chemical compounds may be attached via a polymer spacer or a “linker”. These can be homo- or hetero-bifunctional linkers bearing functional groups such as amino, carbonyl, sulfhydryl, succimidyl, maleimidyl, and isocyanate, (e.g. diaminohexane), ethylene glycobis(sulfosuccimidylsuccinate), disulfosuccimidyl tartarate, dithiobis(sulfosuccimidylpropionate), aminoethanethiol, etc.

The immune-stimulating compounds that may be used according to the present invention include macromolecules, for example, a nucleic acid, a peptide, a peptidoglycan or a lipopolysaccharide; and small molecules (e.g. a low molecular weight synthetic molecule of the imidazoquinoline family, including but not limited to imiquimod, resiquimod and vesatolimod). The immune-stimulating compound may suitably be a double-stranded (ds) RNA, dsDNA or single-stranded (ss) RNA, ssDNA, or a synthetic analog of any of the foregoing (e.g. poly IC, CpG DNA, CpG ODNs, LNAs). In one embodiment, the immune-stimulating compound is an inducer of type 1 interferons (IFN) and the innate immune response. In one embodiment, the immune-stimulating compound is a synthetic (ds) RNA; in one embodiment polyinosinic: polycytidilic acid (PolyIC).

The nanoparticle compositions described herein are suitable for the transport of nucleic acids having 10,000 base pairs (e.g. between 10 and 10,000 nucleotides in length, or between 1000 and 10,000 nucleotides in length.)

In one embodiment, the nanoparticles are covalently or non-covalently linked to one or more TLR agonists. In one embodiment, they are covalently linked through a linking group.

In one embodiment, the TLR agonist is linked non-covalently to a cationic nanoparticle.

As used herein “TLR agonist” refers to any compound, natural or synthetic and analogs thereof, capable of binding to TLRs and inducing a signal transduction. In some embodiments, the compound is selected from the list in Table 1.

TABLE 1
Toll-like receptors and their known natural and synthetic agonists.

| Recep- | Pathogen associated | Endogenous | Synthetic |
|---|---|---|---|
| tor | ligand | ligand | ligand |
| TLR | Triacylated lipopeptides | | Pam3Cys |
| ½ | (Bacteria and | | |
| | Mycobacteria) | | |
| TLR 2 | Peptidoglycan (gram | | CFA, MALP2, |
| | positive bacteria); Bacterial | | Pam2Cys, |
| | lipoprotein, lipoteichoic | | FSL-1, |
| | acid; LPS; GPI-anchor | | Hib-OMPC |
| | proteins; Neisserial porins; | | |
| | Hemagglutinin; | | |
| | phospholipomannan; LAM | | |
| TLR 3 | Viral dsRNA | | Poly I:C; |
| | | | Poly A:U |
| TLR 4 | LPS (gram-negative | Hsp60, Hsp70, | AGP, |
| | bacteria); F-protein (RSV); | fibronectin | MPL A, RC529, |
| | Mannan; | domain A | MDF2β, CFA |
| | Glycoinositolphospholipids; | surfactant | |
| | viral envelope proteins | protein A, | |
| | | hyaluronan; | |
| | | HMGB-1 | |
| TLR 5 | Flagellin | | Flagellin |
| TLR | Phenol-soluble modulin; | | MALP-2, |
| 2/6 | Diacylated lipopeptides; | | Pam2Cys, |
| | LTA; Zymosan | | FSL-1 |
| TLR 7 | Viral ssRNA | Human RNA | Guanosine |
| | | | analogs; |
| | | | imdazo- |
| | | | quinolines |
| | | | (eg. Imiquimod, |
| | | | resiquimod, |
| | | | vesatolimod); |
| | | | Loxoribine |
| TLR 8 | ssRNA from RNA virus | Human RNA | Imidazo- |
| | | | quinolines; |
| | | | Loxoribine; |
| | | | ssPolyU; |
| | | | 3M-012 |
| TLR 9 | Viral dsDNA; Hemozoin; | Human | CpG- |
| | Unmethylated CpG DNA | DNA/chromatin, | oligonucleotides |
| | | LL37-DNA | (CpG-ODN) |

In other embodiments, the nanoparticles are covalently or non-covalently linked to one or more dietary nucleotides, such as IMP, UMP, AMP, CMP, GMP. In one preferred embodiment the dietary nucleotide is IMP. In one embodiment, the dietary nucleotide is covalently linked to an aminated nanoparticle.

The present inventors have demonstrated that being bound to glycogen nanoparticles (whether covalently or non-covalently linked to the nanoparticles) as exemplified herein increases the stability of delivered immune-stimulating compound within the cell. This contributes to enhanced signaling events, leading to a more robust and prolonged innate immune response.

Further, the nanoparticles can be further modified with specific tissue targeting molecules, such as folic acid, antibodies, aptamers, proteins, lipoproteins, hormones, charged molecules, polysaccharides, and low-molecular-weight ligands.

The nanoparticles may also be covalently linked directly or via a spacer, to one or more compounds such as biomolecules, small molecules, therapeutic agents, pharmaceutically active moieties, macromolecules, diagnostic labels, chelating agents, dispersants, surfactants, charge modifying agents, viscosity modifying agents, coagulation agents and flocculants, as well as various combinations of the above.

In one embodiment, the glycogen nanoparticles are covalently linked, directly or via a spacer, to a directing group for targeting to a specific cell type and/or cell compartment. For example, the nanoparticle could be covalently linked to an aptamer, small molecule, receptor ligand, growth factor, antibody or antibody fragment that would target the nanoparticle to a specific tissue or cell type.

Pharmaceutically useful moieties used as modifiers include hydrophobicity modifiers, pharmacokinetic modifiers, biologically active modifiers and detectable modifiers.

Chemical compounds covalently linked to glycogen nanoparticles may have light absorbing, light emitting, fluorescent, luminescent, Raman scattering, fluorescence resonant energy transfer, and electroluminescence properties.

For example, and without limiting the generality of the foregoing, the immunostimulant may comprise glycogen nanoparticles covalently bound to at least one molecule that induces, enhances or amplifies an immune response (e.g. IMP) in an aquaculture species and the phytoglycogen nanoparticles may further be covalently linked to a diagnostic or targeting label. In another example, cationized glycogen nanoparticles are non-covalently linked with a molecule that induces, enhances or amplifies the immune response (e.g. a TLR agonist) and the nanoparticles are further covalently linked to a diagnostic or targeting label.

The innate immune stimulating molecule, covalently or non-covalently linked to glycogen nanoparticles, as described herein provide a surprising and unexpected magnitude of enhancement of the innate immune response.

The combination of properties of glycogen nanoparticles described above together with the feasibility of low production costs, makes the nanoparticle compositions described herein highly suitable for the uses as described herein.

As demonstrated in the Examples, glycogen nanoparticles can carry molecules that enhance an innate immune response across the cell membrane, such molecules being effective within the cells to enhance an innate immune response.

Formulation and Administration

As used herein, “therapeutically effective amount” refers to an amount effective, at dosages and for a particular period of time necessary, to achieve the desired therapeutic result. A therapeutically effective amount of the immunostimulant may vary according to factors such as species, disease state and age. A therapeutically effective amount is also one in which any toxic or detrimental effects are outweighed by the therapeutically beneficial effects.

As used herein “treatment” and grammatical variations thereof refers to administering an immunostimulant of the present invention, in one embodiment to stimulate an immune response to affect an alteration or improvement of a disease or condition, which may include alleviating one or more symptoms thereof, or prophylactically. The treatment may require administration of multiple doses at regular intervals.

Immunostimulants as described herein may also be admixed, encapsulated, or otherwise associated with other molecules, molecule structures or mixtures of compounds and may be combined with a suitable carrier or excipient. The carrier or excipient may be liquid or solid and is selected, with the planned manner of administration in mind, so as to provide for the desired bulk, consistency, etc., when combined with glycogen nanoparticles and other components.

Acceptable carriers may further comprise minor amounts of auxiliary substances such as wetting or emulsifying agents, preservatives or buffers, colour or flavorings, which enhance shelf life or effectiveness.

In an embodiment, the compositions may be lyophilized or spray dried and may be subsequently formulated for administration. For example, in the case of a composition of a nucleic acid immune-stimulating compound non-covalently linked to glycogen nanoparticles, the composition can be lyophilized or spray dried, yielding a product that is stable under storage/transport conditions that would not require cold chain (i.e. no need for refrigeration).

For the purposes of formulating immunostimulant compositions, monodisperse glycogen nanoparticles prepared as taught herein, may be provided in a dried particulate/powder form or may be dissolved e.g. in an aqueous solution. Where a low viscosity is desired, the glycogen nanoparticles may suitably be used in formulations in a concentration of up to about 25% w/w. In applications where a high viscosity is desirable, the nanoparticles may be used in formulations in concentrations above about 25% w/w. In applications where a gel or semi-solid is desirable, concentrations up to about 35% w/w can be used.

In one embodiment, there is provided a food source or food ingredient for aquaculture species comprising an immunostimulant as described herein.

Commercial aquaculture feeds are generally produced by cooking extrusion processing and compositions as described herein may be added to the feed material either before or after extrusion. In one embodiment, the food source or food ingredient comprises pellets, which are cooked with radiant heat or direct heat to a finished edible form. In one embodiment, the composition is applied as a coating or film to a feed. Suitably, the food source comprises about 0.05-1% by weight of the immunostimulant, more preferably 0.05-0.5% by weight of the immunostimulant. Addition of the immunostimulant to feed at the amounts indicated does not significantly alter taste or odour and no detrimental effect on consumption was observed by human feeders in test trials.

In one embodiment, there is provided a method of producing fish or seafood comprising providing to farmed fish or seafood an immunostimulant as described herein.

In one embodiment, the immunostimulants as described herein, which may be provided in the form of a powder or solution are added to an immersion bath at an amount effective to elicit an immune response in an aquaculture species and the aquaculture species is placed in the immersion bath for a time sufficient to elicit the immune response.

In another embodiment, immunostimulants as described herein, which may be in the form of a component of a feed, are orally administered to an aquaculture species in an amount effective to elicit an innate immune response.

The immunostimulants may be administered to prevent or treat an infection in the aquaculture species.

The immunostimulant may be co-administered with a vaccine.

All documents referenced herein are incorporated by reference, however, it should be appreciated that any patent, publication, or other disclosure material, in whole or in part, that is incorporated by reference herein is incorporated only to the extent that the incorporated material does not conflict with definitions, statements, or other disclosure material set forth in this disclosure. As such, and to the extent necessary, the disclosure as explicitly set forth herein supersedes any conflicting material incorporated herein by reference.

It will be understood that numerous modifications thereto will appear to those skilled in the art. Accordingly, the above description and accompanying drawings should be taken as illustrative of the invention and not in a limiting sense. It will further be understood that it is intended to cover any variations, uses, or adaptations of the invention following, in general, the principles of the invention and including such departures from the present disclosure as come within known or customary practice within the art to which the invention pertains and as may be applied to the essential features herein before set forth, and as follows in the scope of the appended claims.

The embodiments of the invention described above are intended to be exemplary only. The scope of the invention is therefore intended to be limited solely by the scope of the appended claims.

### EXAMPLES

### Example 1. Manufacture of Phytoglycogen from Sweet Corn Kernels

1 kg of frozen sweet corn kernels (75% moisture content) was mixed with 2 L of deionized water at 20° C. and was pulverized in a blender at 3000 rpm for 3 min. Mush was centrifuged at 12,000×g for 15 min at 4° C. The combined supernatant fraction was subjected to cross flow filtration (CFF) using a membrane filter with 0.1 μm pore size. The filtrate was further purified by a batch diafiltration using membrane with MWCO of 500 kDa and at RT and diavolume of 6. (Diavolume is the ratio of total mQ water volume introduced to the operation during diafiltration to retentate volume.)

The retentate fraction was mixed with 2.5 volumes of 95% ethanol and centrifuged at 8,000×g for 10 min at 4° C. The retentate was mixed with 2.5 volumes of 95% ethanol and centrifuged at 8,000×g for 10 min at 4° C. The pellet containing phytoglycogen was dried in an oven at 50° C. for 24 h and then milled to 45 mesh. The weight of the dried phytoglycogen was 97 g.

According to dynamic light scattering (DLS) measurements, the phytoglycogen nanoparticles produced had a particle size diameter of 83.0 nm and a polydispersity index of 0.081.

### Example 2. Chemical Modifications of Glycogen Nanoparticles

2.a Quaternary Ammonium Cationization of Phytoglycogen.

Phytoglycogen nanoparticles (PhG) are mixed with aqueous sodium hydroxide solution (1.5-60.0 mmol of NaOH dissolved in 1-5 ml of water/g PhG) and heated to 25 or 45° C. Over the course of 10-120 minutes, 2,3-epoxypropyltrimethylammonium chloride in water (69% solution, 3.07 mL/g PhG) is added. Alternatively, 8.23 mmol of (3-chloro-2-hydroxy-prop-1yl)dimethylalkyl ammonium chloride (alkyl=lauryl, cocoalkyl, stearyl) is stirred with 0.82 mL of 50% NaOH at 45° C. for 5 minutes, and 3.33 ml of 20% aqueous solution of PhG is added. Each reaction is stirred for another 2-6 hours at 45° C. Water (5-9 mL/g PhG) is then added, the mixture is cooled to room temperature and neutralized with 1 M HCl. The product is precipitated and washed in ethanol or hexanes (50-80 mL/g PhG), re-dissolved in water (20 mL/g PhG) and/or saturated NaCl (10 mL) and further purified by dialysis. Freeze-drying affords the product as a white solid. DS (NMR): 0.14-1.46 (Table 2).

2.b Alkylation, Benzylation, or Silylation of Trimethylammonium-Cationized Phytoglycogen.

Trimethylammonium-cationized PhG from Example 2.a is oven-dried at 105° C. for 16 h (silylation) or used as is (alkylation/benzylation). The PhG was dissolved in dry dimethylsulfoxide (20 mL/g PhG) at 80° C. for 1 hour. For alkyl/benzylation reactions, water (0.5 mL) and 50% NaOH (0.042-2.47 mmol/g PhG) is added and stirred vigorously for 10 minutes. Alkyl or benzyl halides (0.51-30.6 mmol/g glycogen) are then added and the mixture is stirred for 2 hours at 60° C., cooled to room temperature and neutralized with glacial acetic acid. For benzylation, the addition of benzyl bromide (0.51 mmol/g PhG) could be performed at 60° C. for 2 hours directly following cationization at 45° C. for 2-6 hours, as a one-pot synthesis. For silylation, the reaction vessel is capped with a rubber septum, cooled to 0° C., and triethylamine (1.19-4.75 mmol/g PhG) is added, followed by dropwise addition of silyl chloride (trimethylsilyl chloride (0.36-1.46 mmol/g PhG), triethylsilyl chloride (0.36 mmol/g PhG)) and stirred overnight at room temperature. For alkylation/benzylation, the crude mixture is extracted several times with diethyl ether/hexanes/ethanol and re-suspended in saturated aqueous NaCl of pH 5-7, then dialysed and freeze-dried; whereas for silylation, the product is precipitated into acetonitrile and washed with hot acetonitrile, then dried overnight at 60° C. and 100 mbar. All are white solids. DS(NMR): alkylation 0.004-1.58; one-pot benzylation: 0.068 for benzyl group and 1.12 for cationic group; silylation 0.19-0.45 (Table 2).

TABLE 2
Reaction parameters and characterization data for 2.a and 2.b.

| | | Alkyl | Cationizing | | | |
|---|---|---|---|---|---|---|
| | aOH | halide | agent | | Hydrodynamic | |
| | (mmol/ | (mmol/ | (mmol/ | | diameter | ζ-potential |
| Substituent | g PhG) | g PhG) | g PhG) | DS_{NMR} | (DLS) | (mV) |
| Ethyl | 0.04 | 0.51 | — | 0.006 | 61.95 | 53.3 |
| Ethyl | 0.17 | 2.04 | — | 0.083 | 64.81 | 51.4 |
| Ethyl | 0.41 | 5.10 | — | 0.66 | 64.38 | 51.3 |
| Dodecyl | 0.04 | 0.51 | — | 0.004 | 76.91 | 57.1 |
| Dodecyl | 0.17 | 2.04 | — | 0.17 | 166 | 33.3 |
| Benzyl | 0.04 | 0.51 | — | 0.052 | 64.57 | 57.9 |
| Benzyl | 0.17 | 2.04 | — | 0.528 | 70.19 | 57.5 |
| Benzyl | 0.41 | 5.10 | — | 1.2 | 78.64 | 62.5 |
| Benzyl | 0.99 | 12.24 | — | 1.58 | 65.61 | 51.6 |
| Benzyl/QUAB151 | 2.5 | 0.51 | 12.4 | 0.068 | 49.98 | 39.9 |
| QUAB 151 | 2.35 | — | 0.28 | 0.34 | 63.81 | 37.4 |
| QUAB 151 | 2.35 | — | 0.56 | 0.47 | 59.18 | 37.6 |
| QUAB 342 | 15.5 | — | 12.5 | 0.88 | 62.74 | 67.7 |
| QUAB 360 | 15.5 | — | 12.5 | 1.02 | 76.58 | 52.6 |
| QUAB 426 | 15.5 | — | 12.5 | 0.136 | 165.7 | 57.7 |

Abbreviations: QUAB, (3-chloro-2-hydroxy-prop-1-yl)dimethylalkyl ammonium chloride (alkyl = lauryl (342), cocoalkyl (360), stearyl (426)), QUAB 151: (3-chloro-2-hydroxy-prop-1yl)trimethylammonium chloride.

2.c TEMPO Oxidized Phytoglycogen.

Phytoglycogen nanoparticles (PhG) are dissolved in glycine buffer (0.05 M, pH 10.20, 33 mL/g PhG). The sample is placed in an ice bath. After 4 hours, a solution of TEMPO (0.04 g/g PhG) in glycine buffer (1.7 mL/g PhG) is added to the reaction mixture. NaBr (0.60 g/g PhG) is then added. After an hour (reaction at 3° C.), NaClO solution (4.52% chlorine) is introduced over the course of 30 minutes (80 mL/g PhG per addition). The reaction mixture is stirred at 0-5° C. for 72 hours, then quenched with anhydrous ethanol (13 mL/g PhG). The mixture is dialyzed (12-14 kDa cut-off) against RO water for 6 cycles, and lyophilized to afford an off-white powder.

### Example 3. Poly IC Bound Phytoglycogen

All solutions and glassware were sterile or autoclaved (121° C. for 30 minutes) and nuclease-free where possible. 1.3 mg of native PhG (prepared as in Example 1) or TEMPO oxidized PhG (Example 2) was dissolved in 0.5 M MES solution, pH 6.4 (0.5 mL). In a separate vial, poly IC (polyinosinic:polycytidylic acid, 4.2 mg) was dissolved in PBS solution (0.5 mL). The PhG and poly IC solutions were stirred at 50° C. for 30 minutes. In a third vial, MES solution at room temperature (3.4 mL) was set aside and 0.1 mL of neat EDC (N-(3-dimethylaminopropyl)N′-ethylcarbodiimide) was added, and the pH was adjusted to 6.7 with NaOH. 1 mL of this EDC/MES solution was added to poly IC-PBS solution, which was transferred into PhG-MES solution. Two controls were also prepared: one without Poly IC, and one without EDC. All reactions were left to stir at 50° C. for 1 hour, then cooled to room temperature. Each reaction mixture was transferred into 100K centrifugal filtration devices (3×0.5 mL) and spun down at 15,000×g for 10 minutes to recover the retentate. Alternatively, samples were dialyzed (MWCO 12-15,000 Da against RO water) for 2 days and lyophilized, or precipitated in ethanol (20 mL) then pelleted by centrifugation (15 min at 7500×g) and dried at 25-50° C. for 6-24 hours.

### Example 4. Cytotoxicity of Glycogen/Phytoglycogen in Cell Cultures

The effects of the glycogen/phytoglycogen nanoparticles on cell viability was analyzed to assess cytotoxicity of the particles. Glycogen/Phytoglycogen nanoparticles were extracted from rabbit liver, mussels, and sweet corn using cold-water and isolated as described in Example 1.

Cationic amphiphilic particles were prepared by functionalizing phytoglycogen with amino-groups, quaternary ammonium groups and alkyl chains of various chain length as described in Example 2 (a) and (b).

Cell lines used: rainbow trout gill epithelium (RTG-2).

To measure changes in cell viability two fluorescence indicator dyes were used, alamar blue (ThermoFisher) and CFDA-AM (Thermofisher); these dyes measure cell metabolism and membrane integrity respectively. For these dyes, more fluorescence indicates more viable cells.

None of the assays detected any cytotoxicity effects in cells after 48 h incubation in the presence of phytoglycogen or its derivatives at concentrations of 0.1-10 mg/ml.

### Example 5. Monodisperse Phytoglycogen Nanoparticles as a Carrier for Double-Stranded RNA

RTgutGC, a rainbow trout gut origin cell line, obtained from N. Bols (University of Waterloo) were used in this study. RTgutGC was routinely cultured at 20 degrees Celsius in 75 cm² plastic tissue culture flasks (BD Falcon, Bedford, Mass.) with Leibovitz's L-15 media supplemented with 10% fetal bovine serum (FBS) and 1% penicillin/streptomycin (P/S).

Poly IC was stored in stock solutions of 10 mg/mL diluted in PBS (Sigma-Aldrich, St Louis, Mo., USA) and stored at −20° C. Poly IC was covalently linked to PhG through Example 3 and made into aliquots of 10 mg/mL PhG covalently linked to 1.44 mg/mL of poly IC. The covalently linked PhG:poly IC was also stored at −20° C.

RTgutGC was seeded at 8×10⁵ cells/well in 6-well plates and left to grow for 24 hours. Cells were treated with media alone (control), 1 ug/mL of poly IC, 1 ug/mL of poly IC covalently linked to 6.875 ug/mL PhG (all using 1×L-15 supplemented with 10% FBS) and incubated at 20° C. for 24 hours. RNA extracted using Trizol, following the manufacturer's instructions exactly. cDNA synthesis using an iScript cDNA synthesis kit (Bio-Rad) was completed using 1 μg of RNA, 4 uL of iScript and up to 20 uL DNA quality water in each reaction. The cDNA was diluted in 1 in 10 in DNA quality water prior to qPCR reactions.

All PCR reactions contained: 2 uL of diluted cDNA, 2× SsoFast EvaGreen Supermix (Bio-Rad), 0.2 um forward primer, 0.2 μM reverse primer and nuclease-free water to a total volume of 10 μL (the housekeeping gene actin primers were at 0.1 μM). The qPCR program was 98° C. 2 mins, 40 Cycles of 98° C. 5 s, 55° C. 10 s and 95° C. for 10 s. A melting curve was completed from 65° C. to 95° C. with a read every 5 s. Gene expression was normalized to the housekeeping gene (03 actin) and expressed as a fold change over the untreated control group.

TABLE 3
Primers designed for qPCR experiments

| | Forward primer | Reverse Primer |
|---|---|---|
| Target | (5′-3′) | (5′-3′) |
| Rainbow | aaaactgtttgat | cgtttcagtct |
| trout IFN-1 | gggaatatgaaa | cctctcaggtt |
| | (SEQ ID NO: 1) | (SEQ ID NO: 2) |
| Rainbow | cggagttcgtct | cccttccacgg |
| trout Mx1 | caacgtct | tacgtctt |
| | (SEQ ID NO: 3) | (SEQ ID NO: 4) |
| Rainbow | gtcaccaactgg | gtacatggcag |
| trout actin | gacgacat | gggtgttga |
| | (SEQ ID NO: 5) | (SEQ ID NO: 6) |
| Rainbow | atggaaaggca | tgagtgggttc |
| trout vig3 | gaggctgtc | tgtaatcagca |
| | (SEQ ID NO: 7) | (SEQ ID NO: 8) |

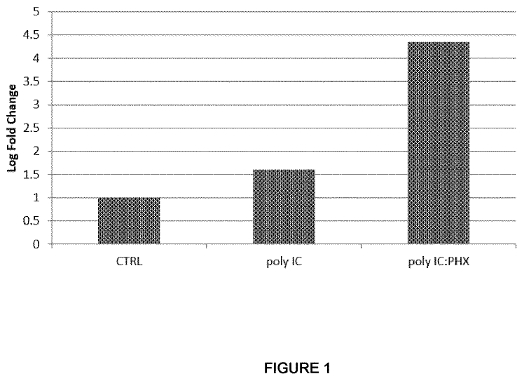

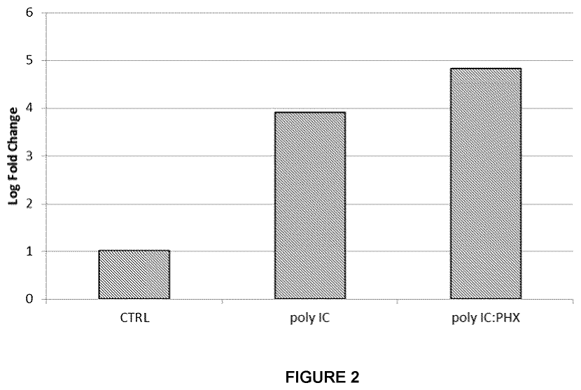

Referring to FIGS. 1 and 2, the results provide evidence that poly IC:PhG induces a stronger antiviral response compared to poly IC alone. This is based on the qRT-PCR data cited, which shows that both IFN1 and vig3 (an ISG) expression levels were higher in the poly IC:phytoglycogen nanoparticle treated cells compared to poly IC alone. This demonstrates that phytoglycogen nanoparticles can be used as a carrier for nucleic acid-based therapeutics that require the nucleic acid to enter the cell.

### Example 6. Binding of Poly IC by Cationic PhG

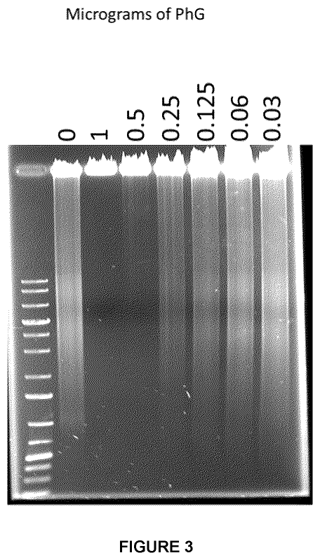

The ability of cationic phytoglycogen nanoparticles (prepared according to Example 2 table 2) (Cat-PhG) to bind poly IC was demonstrated by electrophoretic mobility shift assay (EMSA). Three (3) micrograms of poly IC was mixed with 2-fold dilution series of Cat-PhG with DS 0.88 (Example 2). After a 20-minute incubation at room temperature, samples were separated on a 1% agarose gel, stained with ethidium bromide, and subsequently imaged under UV light. Shown in FIG. 3 are the EMSA gels for Cat-PhG-0.88 (Example 2) demonstrating maximum loading ˜6:1 (poly IC:PhG w/w).

### Example 7. Enhanced Uptake and Intracellular Stability of TLR 3 Agonist Non-Covalently Linked to PhG in Rainbow Trout Gut Cells (RT-Gut)

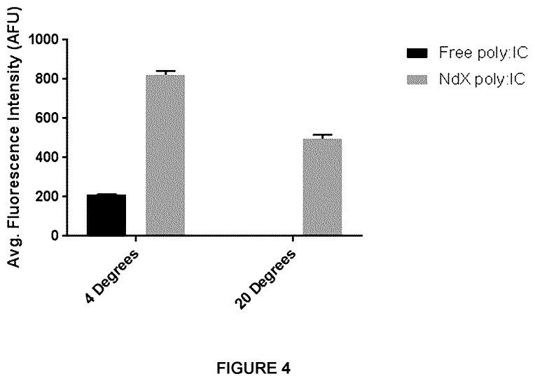

FIG. 4 shows PhG enhances uptake and intracellular stability of TLR 3 agonist in rainbow trout gut cells (RT-gut). Low molecular weight poly IC was labelled with the Ulysis AlexaFluor 546 labelling kit (Invitrogen), then left as “free” poly IC or couple with cationic PhG (0.38) at a 1:1 (w/w) ratio. Free poly IC or PhG coupled poly IC was added to RT-gut cells at a concentration of 1 ug/ml and incubated at 4° C. or 20° C. for 4 hours. Cells were imaged via fluorescent microscopy, and average intracellular fluorescence intensity was measured from 5 randomly selected cells. PhG poly IC treated cells had a higher fluorescence than free poly IC; no fluorescence was detected from free poly IC treated cells for the 20 degree treatment.

### Example 8. Generation of Localized Immune Response after Oral Delivery (Gavage)

Rainbow trout (O. mykiss) weighing approximately 45-65 grams were gently anesthetized with MS-222, and orally gavaged with feed moistened in water, feed moistened with 250 μg HMW poly I:C, or feed moistened with 250 μg of cationic phytoglycogen complexed with 250 μg of HMW poly I:C. At 24 and 48 hours, portions of the proximal, median, and distal intestines were harvested and the RNA was extracted via Trizol (Invitrogen) as per the manufacturer's protocol. One (1) microgram of total RNA was subjected to reverse transcription to generate cDNA (iScript, BioRad). The cDNA was diluted 1:5 and 2 μl was used in a qPCR reaction (EvaGreen, BioRad) with primers specific for O. mykiss actin, ifn1, mx1, and vig3 transcripts. All transcripts were normalized to actin, and quantified relative to “feed-only” fish. Glycogen bound HMW poly I:C induced a greater innate immune response across the entire length of the intestine, compared to poly I:C alone, at both 24 and 48 hours post feeding (FIG. 5-10).

### Example 9. Generation of Systemic Immune Response after Oral Delivery (Gavage)

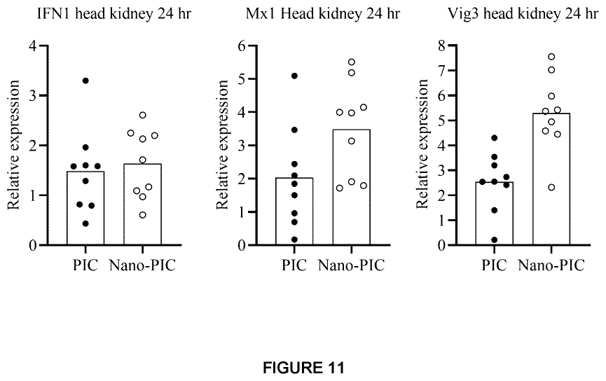

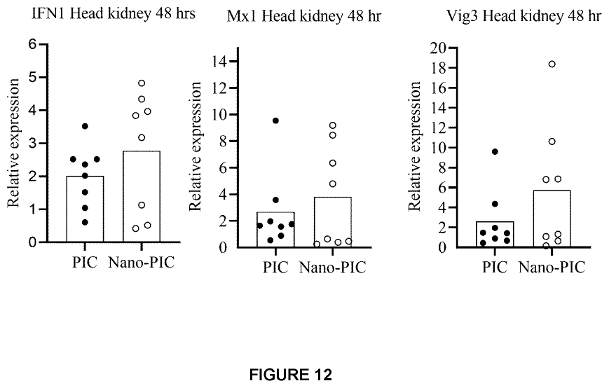

Rainbow trout (O. mykiss) weighing approximately 45-65 grams were gently anesthetized with MS-222, and orally gavaged with feed moistened in water, feed moistened with 250 μg HMW poly I:C, or feed moistened with 250 μg of cationic phytoglycogen complexed with 250 μg of HMW poly I:C. At 24 and 48 hours, portions of the head kidney were harvested and the RNA was extracted via Trizol (Invitrogen) as per the manufacturer's protocol. One (1) microgram of total RNA was subjected to reverse transcription to generate cDNA (iScript, BioRad). The cDNA was diluted 1:5 and 2 μl was used in a qPCR reaction (EvaGreen, BioRad) with primers specific for O. mykiss actin, ifn1, mx1, and vig3 transcripts. All transcripts were normalized to actin, and quantified relative to “feed-only” fish. Glycogen bound HMW poly I:C induced a greater innate immune response systemically, compared to poly I:C alone, at both 24 and 48 hours post feeding (FIG. 11-12).

### Example 10. Formulation of Fish Food Pellets

Commercially available fish feed was ground to a fine powder using a coffee grinder or food processor and mixed with water alone, poly I:C or poly I:C bound to cationic phytoglycogen formulated in water wherepoly I:C and cationic phytoglycogen where combined at a 1:1 ratio. Feed was extruded through a hand held extruder and baked until dry. Dried feed was cut into appropriate-sized pellets. Poly I:C concentrations were added based on estimated feeding at 2% body weight, so that each fish would consume approximately 250 ug of poly I:C per feeding. The final feed comprised 0.0625% by weight pIC, bound to 0.0625% by weight cationic phytoglycogen.

### Example 11. Generation of Localized Immune Response after Oral Delivery (Food Pellets)

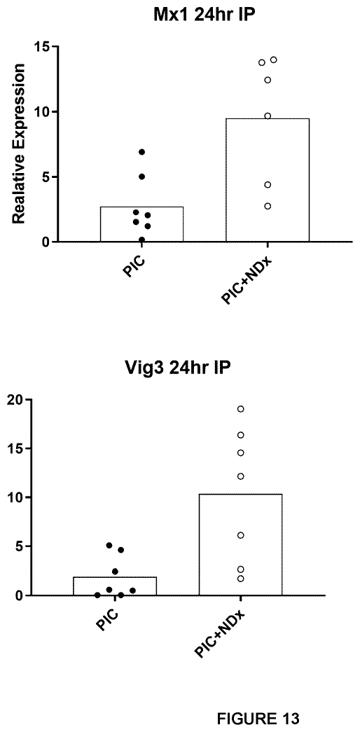

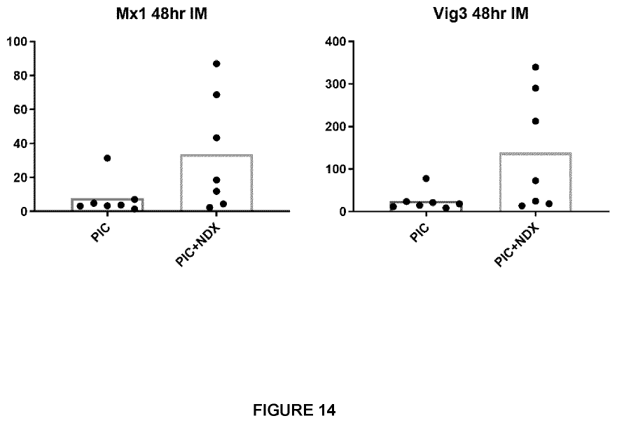

Rainbow trout (O. mykiss) weighing approximately 45-65 grams were fed twice with food pellets formulated with 250 μg HMW poly I:C, or with 250 μg of cationic phytoglycogen complexed with 250 μg of HMW poly I:C. At 24 and 48 hours, portions of the proximal and median intestines were harvested and the RNA was extracted via Trizol (Invitrogen) as per the manufacturer's protocol. One (1) microgram of total RNA was subjected to reverse transcription to generate cDNA (iScript, BioRad). The cDNA was diluted 1:5 and 2 μl was used in a qPCR reaction (EvaGreen, BioRad) with primers specific for O. mykiss actin, ifn1, mx1, and vig3 transcripts. All transcripts were normalized to actin, and quantified relative to “feed-only” fish. Glycogen bound HMW poly I:C induced a greater innate immune response across the entire length of the intestine, compared to poly I:C alone, at both 24 and 48 hours post feeding (FIGS. 13, 14).

### Example 12. Generation of Systemic Immune Response after Oral Delivery (Food Pellets)

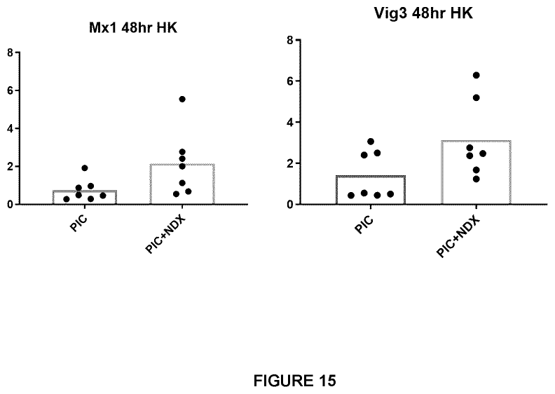

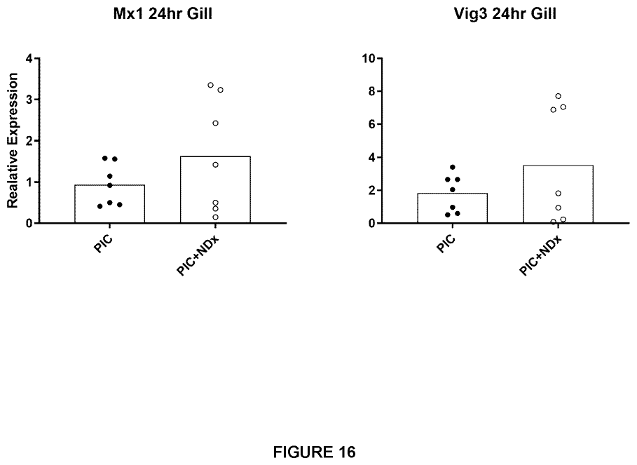

Rainbow trout (O. mykiss) weighing approximately 45-65 grams were fed twice with food pellets formulated with 250 μg HMW poly I:C, or with 250 μg of cationic phytoglycogen complexed with 250 μg of HMW poly I:C. At 24 and 48 hours, portions of the gills head kidney were harvested and the RNA was extracted via Trizol (Invitrogen) as per the manufacturer's protocol and at 24 hours portions of the gill were harvested and the RNA extracted as above. One (1) microgram of total RNA was subjected to reverse transcription to generate cDNA (iScript, BioRad). The cDNA was diluted 1:5 and 2 μl was used in a qPCR reaction (EvaGreen, BioRad) with primers specific for O. mykiss actin, ifn1, mx1, and vig3 transcripts. All transcripts were normalized to actin, and quantified relative to “feed-only” fish. Glycogen bound HMW poly I:C induced a greater innate immune response systemically, compared to poly I:C alone, at both 24 and 48 hours post feeding (FIGS. 15, 16).

### Example 13. Phytoglycogen Nanoparticles Protect dsRNA from Enzymatic Digestion

Five (5) μg of HMW poly I:C was left unbound, or allowed to bind to cationic phytoglycogen nanoparticles for 30 minutes at ratios of 2:1, 1:1, and 0.5:1 phytoglycogen:poly I:C (w/w), then subjected to RNAse iii digestion (BioBasics, Markham ON) as per manufacturer's protocol. Samples were separated via 1% agarose gel and visualized with RedSafe nucleic acid stain (VWR, Mississauga, ON). As shown in FIG. 17, the phytoglycogen nanoparticles had a protective effective on the bound poly I:C against enzymatic digestion.

### Example 14. Dietary Nucleotides Bound to Glycogen Nanoparticles Induce a Greater Innate Immune Response the “Free” Dietary Nucleotides

Rainbow trout intestinal epithelial cells (RT-GUT) were mock-treated or treated with “free” IMP, naked nanoparticle, or IMP covalently bound to aminated nanoparticle. Forty-eight (48) hours post-treatment, RNA was extracted via Trizol (Invitrogen) as per the manufacturer's protocol, and 1 μg of total RNA was subjected to reverse transcription (iScript, BioRad) to generate cDNA. The cDNA was diluted 1:5 and 2 μl was used in a qPCR reaction (EvaGreen, BioRad) with primers specific for O. mykiss actin, ifn1 and mx1 transcripts. All transcripts were normalized to actin, and quantified relative to the mock treated cells. Nanoparticle bound IMP induced significantly more MX1 transcript than “free” IMP.

1. An orally administrable immunostimulant for aquaculture species comprising an immune-stimulating compound covalently or non-covalently linked to glycogen-based polysaccharide nanoparticles having a molecular weight of 10⁶ to 10⁷ Da comprising α-D glucose chains, having an average chain length of 11-12, with 1-4 linkage and branching point occurring at 1→6 and with a branching degree of between 6% and 13%.

2. The orally administrable immunostimulant of claim 1, wherein the immune-stimulating compound is selected from: double-stranded RNA, double-stranded DNA and single-stranded RNA, single-stranded DNA, and synthetic analogs thereof.

3. The orally administrable immunostimulant of claim 2, wherein the immune-stimulating compound is poly IC.

4. The orally administrable immunostimulant of claim 1, wherein the immune-stimulating compound is one or more nucleotides.

5. The orally administrable immunostimulant of claim 2 wherein the immune-stimulating compound comprises between about 50% and 600% by weight relative to the polysaccharide nanoparticles.

6. The orally administrable immunostimulant of claim 2, wherein the immunostimulant has an average particle diameter of between about 10 nm and 500 nm.

7. The orally administrable immunostimulant of claim 1 wherein the immune-stimulating compound is covalently linked to the nanoparticles.

8. The orally administrable immunostimulant of claim 1 wherein the nanoparticles are cationic and the immune-stimulating compound is non-covalently linked to the nanoparticles through electrostatic interactions.

9. The orally administrable immunostimulant of claim 8, wherein the cationic nanoparticles are amine-modified.

10. The orally administrable immunostimulant of claim 1, wherein the nanoparticles are further covalently linked to one or more small molecules for directing the nanoparticles to a type of cell or cellular compartment.

11. The orally administrable immunostimulant of claim 1 in the form of a powder.

12. (canceled)

13. A food source, food ingredient, or a coating for a food source or coating for a food ingredient for aquaculture species comprising the orally administrable immunostimulant of claim 1.

14. The food source of claim 13, wherein the food source, food ingredient or a coating for a food source or coating for a food ingredient comprises between 0.05-1% by weight of the immunostimulant.

15. (canceled)

16. A method of stimulating an innate immune response in an aquaculture species comprising administering to the aquaculture species orally or via immersion bath a therapeutically effective amount of an immunostimulant comprising: an immune-stimulating compound covalently or non-covalently linked to glycogen-based polysaccharide nanoparticles having a molecular weight of 10⁶ to 10⁷ Da comprising α-D glucose chains, having an average chain length of 11-12, with 1-4 linkage and branching point occurring at 1→6 and with a branching degree of between 6% and 13%.

17. The method of claim 16 for preventing or treating a viral, bacterial, parasitic, or fungal infection in the aquaculture species.

18. The method of claim 16, wherein the immunostimulant is administered in conjunction with a vaccine.

19. The method of claim 16 wherein the aquaculture species is a teleost fish.

20. The method of claim 16, wherein the method upregulates expression of one or more of IFN1, VIG-3 and MX1 in the aquaculture species.

21. The orally administrable immunostimulant of claim 5, wherein the glycogen-based polysaccharide nanoparticles are obtained from standard type (su) or sugary extender (se) type sweet corn.

22. The orally administrable immunostimulant of claim 9, wherein the cationic nanoparticles are modified with a short-chain quaternary ammonium compound comprising at least one alkyl moiety having from 1 to 16 carbon atoms, unsubstituted or substituted with one or more N, O, S, or halogen atoms


SEQUENCE LISTING (8 sequences)

SEQ ID NO: 1 - DNA, 25 bp, Artificial Sequence
  primer sequence
aaaactgtttgatgggaatatgaaa

SEQ ID NO: 2 - DNA, 22 bp, Artificial Sequence
  primer sequence
cgtttcagtctcctctcaggtt

SEQ ID NO: 3 - DNA, 20 bp, Artificial Sequence
  primer sequence
cggagttcgtctcaacgtct

SEQ ID NO: 4 - DNA, 19 bp, Artificial Sequence
  primer sequence
cccttccacggtacgtctt

SEQ ID NO: 5 - DNA, 20 bp, Artificial Sequence
  primer sequence
gtcaccaactgggacgacat

SEQ ID NO: 6 - DNA, 20 bp, Artificial Sequence
  primer sequence
gtacatggcaggggtgttga

SEQ ID NO: 7 - DNA, 20 bp, Artificial Sequence
  primer sequence
atggaaaggcagaggctgtc

SEQ ID NO: 8 - DNA, 22 bp, Artificial Sequence
  primer sequence
tgagtgggttctgtaatcagca